(19) United States
(12) Patent Application Publication
(10) Pub. No.: US 2025/0104820 A1
(43) Pub. Date: Mar. 27, 2025

(54) SYSTEM AND METHOD FOR PROVIDING SECURED ACCESS OF ANONYMIZED PATIENT DATASET

(71) Applicant: Partex NV, Eschborn (DE)

(72) Inventor: Abhijit Keskar, Frankfurt (DE)

(21) Appl. No.: 18/474,351

(22) Filed: Sep. 26, 2023

(51) Int. Cl. G16H 10/60; G06F 21/62

(52) U.S. Cl. CPC G16H 10/60; G06F 21/6236

(57) ABSTRACT

A secure system and method have been disclosed to grant authorized access to patient datasets. The system consists of a social security management system, a blockchain, backend systems, and client systems. When a user system requests access to a patient dataset, the social security management system verifies the user's identity. It includes a key generator that generates encryption and decryption keys based on the request, with the encryption key transmitted to the backend system, and the time key and decryption key sent to the user system. The blockchain provides resource locators associated with the request to the user system. The backend system validates timebound requests and encrypts an anonymized patient dataset using the encryption key. The user system, equipped with the decryption key, can decrypt the anonymized dataset and gain access to the patient data. This system ensures secure access and protects patient privacy.

### FIELD OF TECHNOLOGY

Certain embodiments of the disclosure relate to providing secured access of anonymized patient dataset. More specifically, certain embodiments of the disclosure relate to system and method for providing secured access of anonymized patient dataset.

### BACKGROUND

The exchange of patient medical records between healthcare providers and research institutions is essential for providing quality healthcare services. However, the current methods of exchanging medical records are often slow, inefficient, and insecure. Moreover, patient privacy is a significant concern, and the use of traditional methods for sharing medical records can compromise patient confidentiality.

To address these issues, various methods and systems have been proposed in the past, such as electronic health record (EHR) systems, personal health records (PHR) systems, and blockchain-based systems. However, these systems store the data locally thus providing an opportunity of misusing the data. Further, storing the entire data in a blockchain would significantly increase the cost and retrieval time of the dataset. Further limitations include lack of anonymity, lack of security, and the need for a centralized authority to validate the transaction.

Further limitations and disadvantages of conventional and traditional approaches will become apparent to one of skill in the art, through comparison of such systems with some aspects of the present disclosure as set forth in the remainder of the present application with reference to the drawings.

### OBJECT OF THE INVENTION

The objective of the invention is to provide secured access to patient dataset.

Another objective of the invention is to prevent unauthorized access of at least one backend systems.

Yet another objective of the invention is to provide encrypted patient dataset to at least one user system.

Further, another objective of the invention is to allow decryption of the patient dataset locally in environment of the at least one user system.

Furthermore, another objective of the invention is to prevent reuse of resource locator to extract the patient dataset.

Furthermore, another objective of the invention is to provide anonymized patient dataset to the user.

Furthermore, another objective of the invention is to validate a user associated with a social security management system before transmitting the timebound request to the at least one backend system.

Moreover, another objective of the invention is to record the transaction, of retrieving the patient dataset, in a blockchain.

Another objective of the invention is to anonymize the patient dataset before encrypting the patient dataset.

Another objective of the invention is to inactivate a resource locater stored in the blockchain to prevent access to the patient data.

### BRIEF SUMMARY OF THE DISCLOSURE

A system and method for providing secured access of patient dataset, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for providing secured access of anonymized patient dataset, in accordance with an exemplary embodiment of the disclosure.

FIG. 2 is a block diagram that illustrates an exemplary system for patient data registration environment, in accordance with an exemplary embodiment of the disclosure.

FIGS. 3A and 3B depicts a flowchart illustrating exemplary operations for providing secured access of anonymized patient dataset, in accordance with an exemplary embodiment of the disclosure.

### DETAILED DESCRIPTION OF THE DISCLOSURE

Certain embodiments of the disclosure relate to a system and method for providing secured access of patient dataset. The term secured access refers to a system or method that restricts and controls the entry or use of one or more systems associated with social security management system by authorized individuals or entities, while preventing unauthorized access or misuse. The one or more systems comprises at least one user system and at least one backend system. Beneficially, the use of authentication mechanisms such as encryption and other request validation mechanism enables the system to protect sensitive patient dataset from unauthorized access or theft.

Blockchain is a distributed, decentralized ledger technology that records transactions in a secure, transparent, and tamper-proof manner. It is a continuously growing chain of blocks, where each block contains a set of transactions that are validated and linked to the previous block in the chain using cryptography. Once a block is added to the chain, it cannot be altered or deleted, ensuring the integrity and immutability of the data recorded on the blockchain. In the context of the disclosure, the blockchain is configured to store a plurality resource locators associated plurality of backend system associated with plurality of patient identifier, and a record of the retrieval of the patient data from the internal table of the at least one backend system. In an embodiment, the blockchain comprises one or more smart contracts configured to enable transaction between a social security management system and at least one backend system. The one or more smart contracts are configured to store the plurality of resource locator, plurality of patient identifier, and the record of the retrieval in the blockchain. Typically, the plurality of resource locators is of variable length thus directly storing the plurality of resources in the blockchain would make the system unstable. Beneficially, the smart contract appends the plurality of resource locator, plurality of patient identifier, and the record of the retrieval in a defined schema to stabilize the system. Reference in placed to Patent No U.S. Pat. No. 11,586,612B2 and U.S. Pat. No. 11,068,470B2 for more details of the operation of the smart contract, blockchain, data storage and data retrieval. Beneficially, storing the at least one resource locator, at least one patient identifier and a record of the data retrievals in the blockchain ensures security, integrity and immutability of the system.

The social security management system comprises software application and hardware elements that controls access to sensitive patient data and functions within the system. In the context of the current disclosure, the system includes the social security management system, at least one user system, at least one backend system and blockchain. The social security management system verifies the identity of the user and grants access to transact within the system to authorized individuals. The social security management system is communicably connected with the one or more smart contract of the blockchain, the at least one user system and at least one backend system. The social security management system is configured to store the at least one patient identifier and at least one resource locator in the blockchain via the one or more smart contracts. In the context of the disclosure, the social security management system verifies the identity of the user based on social security number of the user to allow the at least one user system to receive the patient dataset from the at least one backend system.

The at least one backend system is the part of the system that provides secured access of patient dataset. The at least one backend system handles data processing, storage, and management, as well as other important functionality that is not directly visible to the end user. In an embodiment it is referred to as the server-side of the application, in contrast to the front-end or client-side which is responsible for displaying the patient dataset to the user. In an embodiment, the at least one backend system comprises of one or more servers that work together to provide the necessary functionality. In an embodiment the at least one backend system comprises web servers, application servers, databases, data processing engines, and messaging systems. The at least one backend system is responsible for storing at least one patient dataset with at least one patient in an internal table, registration of at least one patient with the social security management system, processing timebound requests, validating the request, retrieving the patient dataset from databases, anonymizing the patient dataset, encrypting the anonymized patient dataset, and returning encrypted dataset to the at least one user system. In an embodiment, the at least one backend system is configured to delete the at least one patient dataset from the internal table upon request from the at least one patient. In an embodiment, the at least one backend system does not return any patient dataset from the internal table upon the at least one patient request for opt-out resulting in inactivation of the at least one resource locator.

In some embodiments, the database may be or contain a computer-readable medium, such as a hard disk device, an optical disk device, or a tape device, a flash memory or other similar solid state memory device, or an array of devices, including devices in a storage area network or other configurations for storing the internal table. A computer program product may be tangibly embodied in an information carrier. The information carrier may be a computer-readable or machine-readable medium, such as database. The computer program product may also contain instructions that, when executed, perform one or more methods, such as those described in the disclosure.

The at least one user system is the part of the environment that is visible to the end user and interacts directly with the user. It is often referred to as the client-side of the application, in contrast to the back-end or server-side which handles data processing, storage, and management. The at least one user system includes the graphical user interface (GUI) and other interactive components of the application, such as buttons, menus, and forms, that the user can see and interact with to request the patient dataset. Further, the at least one user system is responsible for decrypting the anonymized patient dataset, presenting the anonymized patient dataset to the user and allowing them to interact with the patient dataset. In an embodiment, at least one user systems or front-end systems include web browsers, mobile apps, and desktop applications. The at least one user system is designed to provide a user-friendly interface for interacting with the application, allowing users to access information, input data, and perform various actions.

The at least one user system is configured to receive a search query from the user and process the search query to identify one or more patient dataset relevant for the user. Specifically, the at least one user system is associated with the user who is interested in accessing the patient dataset. In an embodiment, the at least one user system is configured to present the graphical user interface to the user that allows the user to input the search query. As an example, the user can provide the search query in an input field displayed on the graphical user interface of the at least one user system. The search query may, for example, comprise a set of keywords entered by the user based on his/her area of interest. The search query is parsed to identify one or more possible match. Optionally, the at least one user system is configured to expand the search query to include at least one of: lexical variants for at least one of the one or more query segments, synonyms of at least one of the one or more query segments, abbreviations of at least one of the one or more query segments, word stems of at least one of the one or more query segments. Additionally, optionally, the lexical variants, the synonyms, the abbreviations and/or the word stems are processed into a canonical form (namely, to standardize the one or more query segments).

A user interface may comprise suitable logic, circuitry, and interfaces that may be configured to present the patient dataset. The results are presented in form of an audible, visual, tactile, or other output to the user, such as a researcher, a scientist, a principal investigator, data manager, and a health authority, associated with the at least client system. As such, the user interface may include, for example, a display, one or more switches, buttons, or keys (e.g., a keyboard or other function buttons), a mouse, and/or other input/output mechanisms. In an example embodiment, the user interface may include a plurality of lights, a display, a speaker, a microphone, and/or the like. In some embodiments, the user interface may also provide interface mechanisms that are generated on the display for facilitating user interaction. Thus, for example, the user interface may be configured to provide interface consoles, web pages, web portals, drop down menus, buttons, and/or the like, and components thereof to facilitate user interaction.

Various embodiment of the disclosure provides a system and method for providing secured access of patient dataset. The system comprises a social security management system, a blockchain, at least one backend system and at least one client system. The social security management system is configured to receive a request for patient dataset from an at least one user system and validate a user associated with the request. The social security management system comprises a key generator, wherein the key generator configured to generate at least one encryption key and corresponding at least one decryption key based upon receiving the request for the patient dataset, wherein the at least one encryption key is transmitted to the at least one backend system, and wherein the social security management system is configured to transmit a time key and the at least one decryption key to the at least one user system. The blockchain configured to provide one or more resource locator associated the request to the at least one user system, wherein the at least one user system is configured to communicate the request to the blockchain to receive the one or more resource locator associated the request. The at least one backend system is configured to validate a timebound request from the at least one user system and encrypt an anonymized patient dataset using the encryption key received from the social security management system. The at least one user system is configured to decrypt the anonymized patient dataset based on the received at least one decryption key and access the anonymized patient dataset.

In an embodiment, the at least one backend system is configured to store at least one patient dataset associated with at least one patient in an internal table.

In an embodiment, the at least one backend system is configured to register the at least one patient with the social security management system, wherein the at least one patient registration is based on at least one patient identifier.

In an embodiment, the social security management system is configured to store the at least one patient identifier and at least one resource locator in the blockchain.

In an embodiment, the at least one user system is configured to generate the timebound request based on the request and the time key from the social security management system.

In an embodiment, the at least one backend system retrieves the patient data from the internal table, wherein the at least one backend system anonymizes the patient dataset before encrypting the patient dataset with the encryption key.

In an embodiment, the at least one backend system is configured to store a record of the retrieval of the patient data from the internal table in the blockchain.

In an embodiment, the least one resource locator associated with at least one patient data is inactivated upon request from the at least one patient associated with the at least one patient data.

In an embodiment, the user associated with at least one user system is allowed to access the patient dataset locally with restricted transmission rights.

In accordance with another aspect of the disclosure, a method for providing secured access to anonymized patient dataset is disclosed. The method comprises receiving a request for patient dataset from an at least one user system, validating a user associated with the request, generating at least one encryption key and corresponding at least one decryption key based upon validation of the user associated with at least one user system, transmitting the at least one encryption key to the at least one backend system, and a time key and the at least one decryption key to the at least one user system, providing one or more resource locator associated the request to the at least one user system, wherein the one or more resource locator is provided by the blockchain based on the request, validating the timebound request from the at least one user system, encrypting an anonymized patient dataset using the encryption key received from the key generator, transmitting the encrypted anonymized patient dataset to the at least one user system, decrypting the anonymized patient dataset based on the received at least one decryption key, accessing the patient dataset in the at least one user system.

In accordance with the embodiment, the method comprises storing at least one patient dataset associated with at least one patient in an internal table in the at least one backend system.

In accordance with the embodiment, the method comprises, registration of the at least one patient with the social security management system, wherein the registration is based on at least one patient identifier.

In accordance with the embodiment, comprises storing the at least one patient identifier and at least one resource locator in the blockchain

In accordance with the embodiment, the method comprises generating the timebound request based on the request from the at least one user system and the time key from the social security management system.

In accordance with the embodiment, the method comprises retrieving the patient data from an internal table, wherein the at least one backend system anonymizes the patient dataset before encrypting the patient dataset with the encryption key.

In accordance with the embodiment, the method comprises storing a record of the retrieval of the patient data from the internal table in the blockchain.

In accordance with the embodiment, the method comprises inactivating the least one resource locator associated with at least one patient data upon request from the at least one patient associated with the at least one patient data

In accordance with the embodiment, the method comprises allowing access to the patient dataset locally with restricted transmission rights to the user of the at least one user system.

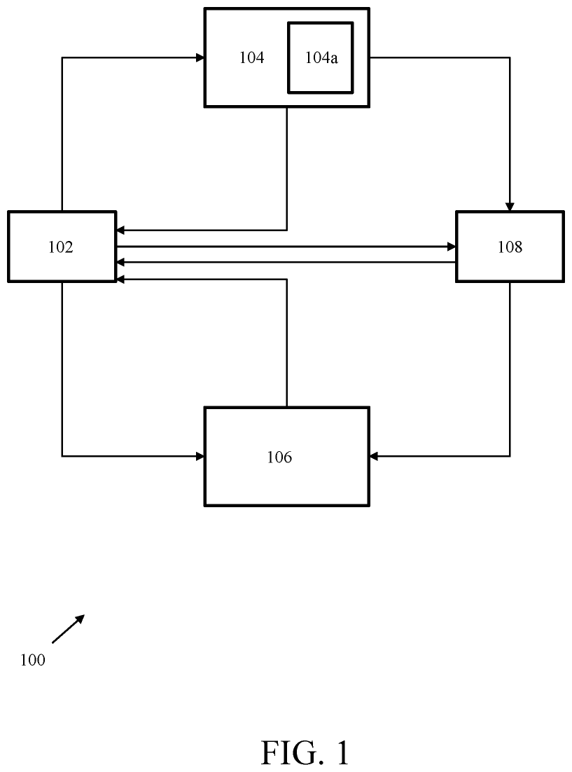

FIG. 1 is a block diagram that illustrates an exemplary system for providing secured access to patient dataset. Referring to FIG. 1, a system 100 comprises at least one user system 102, a social security management system 104, a blockchain 106, and at last one backend system 108. The social security management system 104 comprises a key generator 104a configured to generate at least one encryption key and at least one decryption key. The at least one user system 102, the social security management system 104, the blockchain 106, and the at last one backend system 108 is communicable coupled via the communication network.

The communication network may be any kind of network, or a combination of various networks, and it is shown illustrating exemplary communication that may occur between the one or more system, the social security management system and blockchain. For example, the communication network may comprise one or more of a cable television network, the Internet, a satellite communication network, or a group of interconnected networks (for example, Wide Area Networks or WANs), such as the World Wide Web. Although one mode of communication network the communication network is shown, the disclosure is not limited in this regard. Accordingly, other exemplary modes may comprise uni-directional or bi-directional distribution, such as packet-radio, and satellite networks.

The at least one user system 102 is configured to provide an authorized user to access one or more patient dataset based upon the validation of the user and automated security clearances. The at least one user system 102 comprises a GUI configured to receive user information and information related to identify the patient dataset from a plurality of patient dataset. In an embodiment, the user associated with the at least one user system 102 searches for one or more patient data on the GUI based on one or more search. The at least one user system 102 is configured to communicate a request associated with the user to the social security management system 104, wherein the request comprises at least one patient dataset identifier. The at least one user system 102 is configured to receive at least one decryption key and a time key upon verification of the user from the social security management system 104. Further, the at least one user system 102 is configured to communicate the request to the one or more smart contract of the Blockchain 106. The at least one user system 102 is configured to communicate with the at least one backend system 108 for receiving the patient dataset based upon one or more resource location from the one or more smart contact of the blockchain 106. The at least one user system 102 is configured to transmit a timebound request to the at least one backend system 106. The at least one user system 102 is configured to receive an encrypted anonymized patient dataset within a pre-determined timeframe provided the user and the request receives security clearances from the social security management system 104 and the at least one back-end system 106. The at least one user system is configured to decrypt the encrypted anonymized patient dataset based on the received at least one decryption key to access the patient dataset. In an embodiment, the user associated with the at least one user system is allowed to access the patient dataset locally with restricted transmission rights. In an embodiment, the at least one user system would receive a null result if the at least one patient associated with the at least one patient dataset has opted-out.

The social security management system 104 is configured to receive the request associated with the user and at least one patient dataset identifier from the at least one user system 102. In embodiment, the social security management system is configured to store the at least one patient identifier and at least one resource locator in the blockchain 108 via the one or more smart contracts. The social security management system 104 is configured to validate the user associated with the request based on social security number of the user. The social security management system 104 is configured to initiate a key generator 104a upon validation of the user. The key generator 104a configured to generate at least one encryption key and corresponding at least one decryption key based upon the request. The social security management system 104 is configured to transmit the at least one encryption key to the at least one backend system 108, and the time key and the at least one decryption key to the at least one client system 102. Beneficially, the social security management system 104 enables the access of the patient dataset to authorized users only. Further beneficially, the social security management system 104 allows the transaction of the patient dataset from the at least one backend system 108 to at least one user system 102 in a timebound manner. In an example, the social security management system 104 is government-controlled software application. Further beneficially, the social security management system 104 ensures authenticity of the entire system.

The blockchain 106 is configured to provide at least one resource locator associated the at least one backend system to the at least one user system based on the request from the at least one user system. In an embodiment, the blockchain 106 comprises one or more smart contracts configured to enable transaction between a social security management system 104 and at least one backend system 108. The one or more smart contracts are configured to store the plurality of resource locator, plurality of patient identifier, and the record of the retrieval in the blockchain 106. Typically, the plurality of resource locators is of variable length thus directly storing the plurality of resources in the blockchain 106 would make the system 100 unstable. Beneficially, the smart contract appends the plurality of resource locator, plurality of patient identifier, and the record of the retrieval in a defined schema to stabilize the system 100. Reference in placed to Patent No U.S. Pat. No. 11,586,612B2 and U.S. Pat. No. 11,068,470B2 for more details of the operation of the smart contract, blockchain 106, data storage and data retrieval. In an embodiment, the blockchain 106 is configured to store at least one resource locator associated with a patient dataset and at least one patient identifier associated with a plurality of patient data, wherein the at least one patient identifier comprise information to patient's social security identification number. In an embodiment, the blockchain 106 is configured to store a record of the retrieval of the patient data from the internal table. Beneficially, the blockchain 106 allows the system to retain its integrity and immutability of the transaction.

The at least one backend system 108 is configured to validate a timebound request and encrypts an anonymized patient dataset using the encryption key. The at least one backend system 108 is configured to receive the timebound request and the at least one encryption key from the social security management system 104. In an embodiment, the at least one backend system is configured to store at least one patient dataset associated with at least one patient in an internal table. In an embodiment, the at least one backend system is configured to register at least one patient with the social security management system, wherein the at least one patient registration is based on at least one patient identifier. The at least one back-end system 108 is configured to validate the timebound request based upon receipt of the timebound request from the at least one user system and receipt of the timebound request from the at least one user system within a pre-defined time period. In an example, the time key may define pre-defined time period of 2-5 sec. In an embodiment, the at least one backend system is configured to compare the timebound request from the at least one user system comprising the request and the time key with the time associated with the receipt of the encryption key from the social security management system 104 to validate the transaction. Beneficially, the timebound prevents reuse of the one or more resource locator post the pre-defined time period. Upon successful validation of the timebound request, the at least one back-end system 108 is configured to retrieve the patient data from the internal table. The at least one backend system 108 is configured to anonymize the retrieved patient dataset before encrypting the patient dataset with the at least one encryption key received from the social security management system 104. In an embodiment, the at least one backend system 108 is configured to skip personally identifiable information of the at least one patient before encrypting the patient dataset. Beneficially, skip personally identifiable information of the at least one patient ensures anonymity of the at least one patient associated with the patient dataset.

In an embodiment, a patent associated with the patient dataset

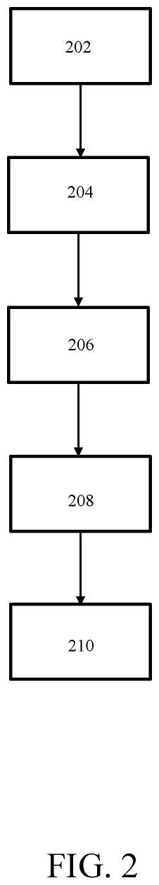

FIG. 2 is a flowchart that illustrates a method for patient data registration environment.

At step 202, a patient visits a hospital or clinic.

At step 204, the staff enters patient dataset in the at least back-end system 108.

At step 206, the at least one backend system 108 initiates registration in the social security management system 104.

At step 208, the social security management system 104 generates an OTP on the registered phone of the patient. Upon validation of the OTP the social security management system 104 registers the patient. The social security management system 104 maps the resource location of the at least one backend system against the patient's social security identification number in the blockchain.

At step 210, upon successful registration and mapping the hospital and the patient receives confirmation.

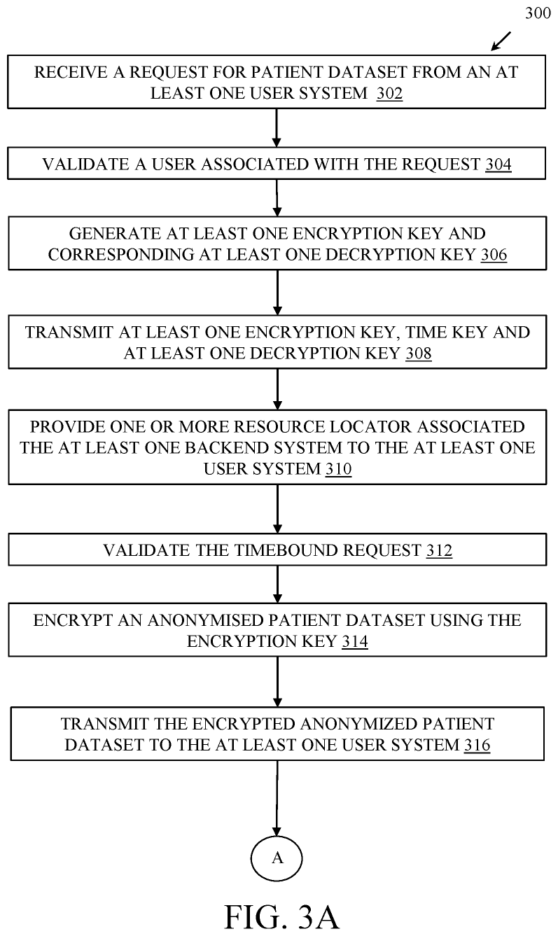

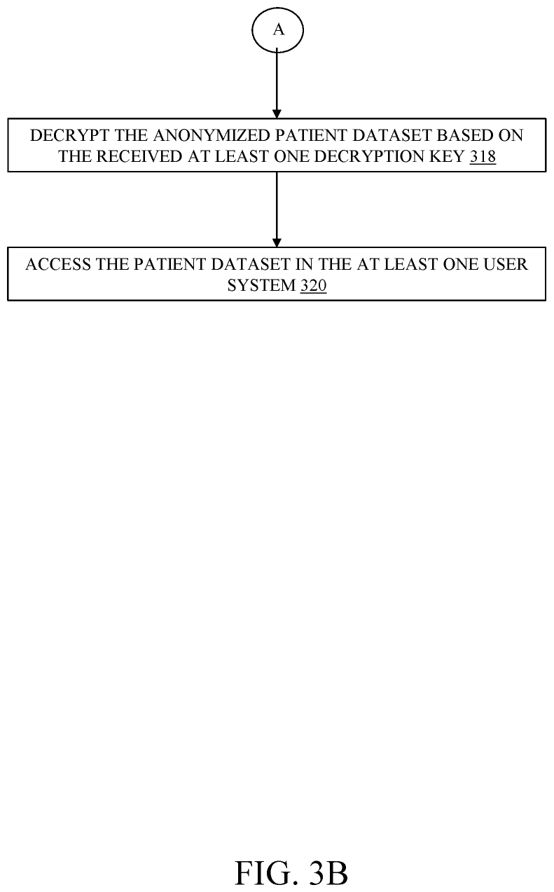

FIGS. 3A and 3B depict flowcharts illustrating exemplary operations for for providing secured access to patient dataset. FIGS. 3A and 3B respectively, are described in conjunction with FIG. 1.

At step 302, a request for patient dataset is received by a social security management system 104 from at least one user system 102.

At step 304, the social security management system 104 is configured to validate a user associated with the at least one user system 102.

At step 306, a key generator 104a is configured to generate at least one encryption key and corresponding at least one decryption key.

At step 308, the social security management system 104 is configured to transmit at least one encryption key to the at least one user system 102 and time key and at least one decryption key to the at least one backend system 108.

At step 310, the blockchain 106 is configured to provide one or more resource locator associated with the at least one backend system to the least one user system.

At step 312, the at least one backend system 108 is configured to validate the timebound request.

At step 314, the least one backend system 108 is configured to encrypt an anonymized patient dataset using the at least one encryption key received from the social security management system 104.

At step 316, the at least one backend system 108 is configured to transmit the encrypted anonymized patient dataset to the at least one user system 102.

At step 318, the at least one user system 102 is configured to decrypt the anonymized patient dataset based on the received at least one decryption key.

At step 320, the user accesses the patient dataset in the at least one user system 102.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for determining combination drug and use in pancreatic cancer treatment.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A system for providing secured access of anonymized patient dataset, comprising:
a social security management system configured to
receive a request for patient dataset from an at least one user system; and
validate a user associated with the request, wherein the social security management system comprises of key generator configured to generate at least one encryption key and corresponding at least one decryption key based upon the request, and
wherein the at least one encryption key is transmitted to at least one backend system, and
wherein the social security management system is configured to transmit a time key and the at least one decryption key to the at least one user system;
a blockchain configured to provide one or more resource locator associated the request to the at least one user system, wherein the at least one user system is configured to communicate the request to the blockchain to receive the one or more resource locator associated the request;
the at least one backend system configured to validate a timebound request from the at least one user system and encrypts an anonymized patient dataset using the encryption key received from the social security management system; and
the at least one user system configured to decrypt the anonymized patient dataset based on the received at least one decryption key and access the anonymized patient dataset.

2. The system as claimed in claim 1, wherein the at least one back-end system is configured to store at least one patient dataset associated with at least one patient in an internal table.

3. The system as claimed in claim 2, wherein the at least one back-end system is configured to register the at least one patient with the social security management system, wherein the at least one patient registration is based on at least one patient identifier.

4. The system as claimed in claim 3, wherein the social security management system is configured to store the at least one patient identifier and at least one resource locator in the blockchain.

5. The system as claimed in claim 1, wherein the at least one user system is configured to generate the timebound request based on the request and the time key from the social security management system.

6. The system as claimed in claim 1, wherein the at least one back-end system retrieves the patient data from the internal table, wherein the at least one backend system anonymizes the patient dataset before encrypting the patient dataset with the encryption key.

7. The system as claimed in claim 1, wherein the at least one back-end system is configured to store a record of the retrieval of the patient data from the internal table in the blockchain.

8. The system as claimed in claim 1, wherein the least one resource locator associated with at least one patient data is inactivated upon request from the at least one patient associated with the at least one patient data.

9. The system as claimed in claim 1, wherein the user associated with at least one user system is allowed to access the patient dataset locally with restricted transmission rights.

10. A method for providing secured access to anonymized patient dataset, the method comprising:
receiving a request for patient dataset from an at least one user system,
validating a user associated with the request,
generating at least one encryption key and corresponding at least one decryption key based upon validation of the user associated with at least one user system,
transmitting the at least one encryption key to the at least one back-end system, and a time key and the at least one decryption key to the at least one user system,
providing one or more resource locator associated the request to the at least one user system, wherein the one or more resource locator is provided by the blockchain based on the request,
validating the timebound request from the at least one user system,
encrypting an anonymized patient dataset using the encryption key received from the key generator,
transmitting the encrypted anonymized patient dataset to the at least one user system,
decrypting the anonymized patient dataset based on the received at least one decryption key,
accessing the patient dataset in the at least one user system.

11. The method as claimed in claim 10, comprise storing at least one patient dataset associated with at least one patient in an internal table in the at least one backend system.

12. The method as claimed in claim 11, comprises registration of the at least one patient with the social security management system, wherein the registration is based on at least one patient identifier.

13. The method as claimed in claim 12, comprises storing the at least one patient identifier and at least one resource locator in the blockchain.

14. The method as claimed in claim 10, comprises generating the timebound request based on the request and the time key from the social security management system.

15. The method as claimed in claim 10, comprises retrieving the patient data from the internal table, wherein the at least one backend system anonymizes the patient dataset before encrypting the patient dataset with the encryption key.

16. The method as claimed in claim 10, comprises storing a record of the retrieval of the patient data from the internal table in the blockchain.

17. The method as claimed in claim 10, comprises inactivating the least one resource locator associated with at least one patient data upon request from the at least one patient associated with the at least one patient data.

18. The method as claimed in claim 10, comprises allowing access to the patient dataset locally with restricted transmission rights.
